(19) United States
(12) Patent Application Publication
(10) Pub. No.: US 2026/0225035 A1
(43) Pub. Date: Aug. 6, 2026

(54) MICROPLASMA CATALYTIC REACTOR

(71) Applicant: Dyson Technology Limited, Malmesbury, Wiltshire (GB)

(72) Inventors: Muhammad Arsalan ASHRAF, Bath (GB); Yihan LIU, Bristol (GB); Gavin Alexander WITHERS, Bath (GB); Ian Jeffrey STEPHENSON, Huddersfield (GB); Kenneth Mark ARMSTRONG, Bristol (GB)

(21) Appl. No.: 19/159,759

(22) PCT Filed: Feb. 14, 2024
(86) PCT No.: PCT/IB2024/051370

§ 371 Date: Aug. 26, 2025

(30) Foreign Application Priority Data
Feb. 27, 2023 (GB) 2302844.2

(51) Int. Cl. B01D 53/32; B01J 19/00; B01J 19/08

(52) U.S. Cl. CPC B01D 53/32; B01J 19/0093; B01J 19/088; B01D 2257/708; B01D 2258/06; B01D 2259/818; B01J 2219/0809; B01J 2219/0815; B01J 2219/0894; B01J 2219/2427

(57) ABSTRACT

Provided is a microplasma catalytic reactor for treatment of ambient air by decomposition of VOCs in the ambient air. The reactor includes a first electrode member comprising an electrically conducting core and a dielectric coating, and a second electrode member disposed with respect to the first electrode member so as to generate a plasma between the first and second electrode members on the application of a plasma generation voltage between the first and second electrode members such that VOCs in the ambient air in the reactor are decomposed to form VOC decomposition products, and plasma by-products are formed. The dielectric coating further includes a catalyst that catalyses the decomposition of one or more of the VOCs, the VOC decomposition products, and plasma by-products.

### FIELD OF THE INVENTION

The present invention relates to a microplasma catalytic reactor, an air purification and a method of operating a microplasma catalytic reactor.

### BACKGROUND

The negative impact of pollutants, such as VOCs, on human health in indoor environments is known. Many indoor pollutants found in residential settings are organic chemicals and span a wide range of functional groups, size and potential chemistries. These can be categorised as volatile organic compounds (VOCs) and volatile inorganic compounds (VICs). VOCs that humans are commonly exposed to in indoor environments include alcohols, aldehydes, aromatic compounds, ketones, alkanes and alkenes.

Various techniques have been investigated for the removal of VOCs from air, such as adsorption, catalytic thermal oxidation, cold plasma and photo-catalysis.

It is known that VOCs readily break down in the presence of plasma (an ionised gas phase). However, the formation of plasma by-products such as ozone (O₃) and nitrous oxides (NOₓ) within the plasma, as well as the formation of partially-oxidised VOCs, amongst other by-products, is a major obstacle in employing this technology indoors.

WHO guideline values for NOₓ are 10 μg/m³ (5.3 ppb) annual mean and 25 μg/m³ (13.3 ppb) 24-hr mean. Based on the toxicology profile of NOₓ, the European Commission Scientific Committee (ECSC) recommends an occupational exposure limit of 2 ppm (time-weighted average over 8 hours). The OSHA recommends an immediately dangerous to life or health limit of 100 ppm and a permissible exposure limit of 25 ppm (time-weighted average over 8 hours). The WHO guideline values for ozone are 100 μg/m³ (51 ppb) 8 hr daily maximum and 60 μg/m³ (31 ppb) as an average of daily maximum time-weighted average over 8 hours in six months of peak season O₃ concentration.

A microplasma dielectric barrier discharge (DBD) reactor is a reactor technology to produce plasma at lower voltages (about 1 kV) than typical and in small discharge gaps (<1 mm) with low free-electron energy. Generating high-energy free electrons increases the possibility of generating various reactive species within the reactor that then lead to NOₓ and O₃ formation. Therefore, microplasma DBD reactors have the potential to reduce NOₓ formation when using plasma to treat a VOC-containing airflow. However, conversion of VOCs, O₃ generation, partially-oxidised VOC generation and NOₓ generation all remain problems with known microplasma DBD reactors.

The present invention has been devised in light of the above considerations.

### SUMMARY OF THE INVENTION

In a first aspect of the present disclosure there is provided a microplasma catalytic reactor for treatment of ambient air by decomposition of VOCs in the ambient air. The reactor comprises: a first electrode member comprising an electrically conducting core and a dielectric coating; and a second electrode member disposed with respect to the first electrode member so as to generate a plasma between the first and second electrode members on the application of a plasma generation voltage between the first and second electrode members such that, in use, VOCs in the ambient air in the reactor are decomposed such that VOC decomposition products and plasma by-products are formed. The dielectric coating further comprises a catalyst that catalyses the decomposition of one or more of: the VOCs; the VOC decomposition products; and the plasma by-products. Advantageously, such a microplasma catalytic reactor is able to effectively decompose VOCs in the ambient air whilst limiting the quantity of plasma by-products and partially-oxidised VOCs released by the reactor.

The catalyst contained within the dielectric coating may comprise one or more of: a metal oxide, a mixed metal oxide, a noble metal, a noble metal-metal oxide composite, and a noble metal-mixed metal oxide composite. Advantageously, this allows the composition of the catalyst within the dielectric coating to be tailored to the quantity and/or type of VOCs contained within the ambient air that require decomposition and/or the quantity and/or type of plasma by-products generated within the reactor.

The catalyst may comprise one or more of: Ag, Pt, Pd, Rh, Ni, Cu, Mo, Co, Mg and Ti. Advantageously, a catalyst comprising one or more of the above elements is effective in catalysing the decomposition of one or more of the VOCs, the VOC decomposition products, and the plasma by-products.

The first electrode and second electrode may be arranged upstream and downstream of each other with respect to an airflow path through the reactor. Advantageously, a reactor having such a geometry is able to generate a plasma that spans a range of residence times within the reactor and therefore catalyse a range of compounds within the plasma.

The second electrode member of the first aspect may comprise an electrically conducting core and a dielectric coating. Advantageously, both electrodes having the structure of an electrically conducting core and a dielectric coating reduces the likelihood of arcing between the electrode members and thus reduces the likelihood of formation of a large quantity of plasma arc by-products.

The dielectric coating of the second electrode member may also further comprise a catalyst that catalyses the decomposition of one or more of: the VOCs; the VOC decomposition products; and the plasma by-products. This is advantageous in providing a greater surface area of catalyst to catalyse the decomposition of these compounds.

The dielectric coating of the first electrode member and/or the dielectric coating of the second electrode member may comprise a first catalyst and a second catalyst. Advantageously, the presence of two catalysts on an electrode member may allow each catalyst to be more specialised in catalysing the decomposition of a certain compound without overly compromising the catalysis of other compounds within reactor.

Alternatively, where both the first and second electrode members comprise a dielectric coating comprising a catalyst, the dielectric coating of the first electrode member may comprise a first catalyst and the dielectric coating of the second electrode may comprise a second catalyst. The first catalyst may be different from the second catalyst. Advantageously, this can allow the catalytic efficiency of the reactor to be improved by tailoring the catalyst on a given electrode to the compounds in the vicinity of that electrode during operation of the reactor.

Where the first and second electrode members are arranged upstream and downstream of each other with respect to the airflow path through the reactor, the first catalyst may only be applied to the downstream electrode. Advantageously, such a configuration may allow for efficient use of the first catalyst as, with respect to the midpoint between the two electrode members, the catalyst is positioned in the direction the reactive species generated in the plasma will travel through the reactor.

The electrically conducting cores of each electrode member may comprise one or more of stainless steel, aluminium, brass, iron or copper. Advantageously, the composition of the electrically conducting cores can be tailored based on the desired conductivity, cost, weight, and ductility of the electrically conducting cores.

The dielectric coating of an electrode member may be porous or non-porous. For porous coating, any catalyst that the dielectric coating comprises may be disposed, at least in part, on internal surfaces of the pores of the dielectric coating. Advantageously, such a structure can provide an increased surface area for the catalysis of the decomposition reactions occurring within the reactor.

Where the dielectric coating of an electrode member is porous and a catalyst is to be disposed, at least in part, on internal surfaces of the pores of the dielectric coating, the catalyst may be disposed on internal surfaces of the pores using an impregnation technique, such as, incipient wetness impregnation technique, wet impregnation technique. Advantageously, incipient wetness impregnation technique is technically simple, low cost, and produces limited amounts of waste, whilst also ensuring that catalyst is disposed within the pores of the substrate, meaning the overall thickness and uniformity of the coating is not impacted by the deposition of catalyst.

Porous dielectric coating may present inhomogeneity in terms of wide range of cracks and pores into the film, may connected internally down to metal electrode, which may alter the discharge characteristics and breakdown mechanism. The impregnation of dielectric film with a catalyst provides additional benefit of pore/crack sealing, in this way, the porosity of coating film can be controlled from a relatively porous to non-porous, which is critical in maintaining the desired electrical, mechanical, and chemical characteristics,

The dielectric coating of an electrode member may comprise a metal oxide. The metal oxide may be one or more of MnO₂, Al₂O₃, CeO₂, SiO₂, and TiO₂. Alternatively, the dielectric coating of an electrode member may comprise activated carbon or a zeolite. Advantageously, such materials have a high surface area and can also act as a catalyst in the presence of a plasma. Nobel metal catalysts, one or more of Au, Ag, Tu, Rh, Pd and Pt, can be impregnated in the metal oxide coating layer.

The reactor according to the first aspect of the present disclosure may comprise a plurality of first electrode members and a plurality of second electrode members, with each pair of adjacent first electrode members interposed by a second electrode member and each pair of adjacent second electrode members interposed by a first electrode member. Advantageously, such a reactor can process a larger volume of air than a reactor having only a single first electrode member and a single second electrode member whilst maintaining the conversion rate of the reactor by increasing the residence time of the air within the reactor.

The reactor according to the first aspect of the present disclosure may further comprise a power supply connected to each first electrode member and/or each second electrode member. The power supply so connected may be configured to deliver pulsed DC to each electrode member to which it is connected such as to generate a plasma between a first electrode member and its adjacent second electrode member. By providing pulsed DC to the electrode members connected to the power supply, the energy consumption of the reactor can be reduced in comparison to using non-pulsed DC and additional control variables are provided for controlling the plasma discharge within the reactor.

The reactor according to the first aspect of the present disclosure may further comprise a third electrode member, the third electrode member being interposed between the first electrode member and the second electrode member. The third electrode member may incorporate one or more of the optional features of the first and/or second electrode members set out above.

The power supply may be configured such that the parameters of the pulsed DC that the power supply is configured to deliver to each electrode member to which it is connected can be adjusted independently of the other electrode members to which it is connected. Advantageously this allows the plasma discharge between a pair of adjacent electrode members to be controlled independently of the plasma discharge between other pairs of adjacent electrode members.

The reactor may be configured such that the pulsed DC received by each electrode member connected to the power supply: generates an electric field strength with a voltage greater than or equal to [0.3 kV] and less than or equal to [1.9 kV] between that electrode member and an adjacent electrode member; has a pulse frequency greater than or equal to 0.5 kHz and less than or equal to 100 kHz; and has a pulse width greater than or equal to 0.6 μs and less than or equal to 50 μs. Advantageously, the reactor being configured to provide such pulsed DC to each electrode member that the power supply is connected to facilitates the effective decompose VOCs in the ambient air whilst limiting the quantity of plasma by-products and partially-oxidised VOCs released by the reactor.

In a reactor comprising a power supply configured to deliver pulsed DC to each electrode member to which it is connected, the pulsed DC received by each electrode member may have a voltage greater than or equal to 0.3 kV. The pulsed DC received by each electrode member may have a voltage less than or equal to 1.9 kV. The pulsed DC received by each electrode member may have a voltage greater than or equal to 0.8 kV. The pulsed DC received by each electrode member may have a voltage less than or equal to 1.0 kV. The pulsed DC received by each electrode member may have a voltage greater than or equal to 0.88 kV. The pulsed DC received by each electrode member may have a voltage less than or equal to 0.92 kV. Providing pulsed DC with such a voltage to each electrode member is beneficial in generating a plasma to catalyse the decomposition of VOCs whilst limiting the presence of plasma by-products in air exiting the reactor.

In a reactor comprising a power supply configured to deliver pulsed DC to each electrode member to which it is connected, the pulsed DC received by each electrode member may have a current amplitude greater than or equal to 0.01 A. The pulsed DC received by each electrode member may have a current amplitude less than or equal to 8 A. The pulsed DC received by each electrode member may have a current amplitude greater than or equal to 0.2 A. The pulsed DC received by each electrode member may have a current amplitude greater than or equal to 0.3 A. The pulsed DC received by each electrode member may have a current amplitude greater than or equal to 0.4 A The pulsed DC received by each electrode member may have a current amplitude less than or equal to 0.5 A. Providing pulsed DC with such a current amplitude to each electrode member is beneficial in generating a plasma to catalyse the decomposition of VOCs whilst limiting the presence of plasma by-products in air exiting the reactor.

In a reactor comprising a power supply configured to deliver pulsed DC to each electrode member to which it is connected, the pulsed DC received by each electrode member may have a pulse width greater than or equal to 0.05 μs. The pulsed DC received by each electrode member may have a pulse width greater than or equal to 3 μs. The pulsed DC received by each electrode member may have a pulse width less than or equal to 10 μs. The pulsed DC received by each electrode member may have a pulse width less than or equal to 50 μs. The pulsed DC received by each electrode member may have a pulse width less than or equal to 15 μs. The pulsed DC received by each electrode member may have a pulse width less than or equal to 5 μs. Providing pulsed DC with such a pulse width to each electrode member is beneficial in generating a plasma to catalyse the decomposition of VOCs whilst limiting the presence of plasma by-products in air exiting the reactor.

In the reactor according to the first aspect of the present disclosure, each electrode member may be a perforated plate. Advantageously, providing the electrode members as perforated plates facilitates a large amount of contact between the ambient air and electrode members, increasing the conversion of the reactor. Moreover, perforated plate structures are easy to manufacture and provide electrode members with a high surface-area-to-volume ratio, reducing redundant material within the electrode member. The perforated plates may have an open area percentage greater than or equal to 10%. The perforated plates may have an open area percentage less than or equal to 35%. The perforated plates may have an open area percentage greater than or equal to 22%. The perforated plates may have an open area percentage less than or equal to 90%. The perforated plates may have an open area percentage greater than or equal to 24.5%. The perforated plates may have an open area percentage less than or equal to 25.5%. Advantageously, the open area percentage may be set such as to balance the pressure drop across the reactor with the surface area of the electrode members that can be used for catalysis and plasma generation.

Where the electrode members are plate-shaped and offset from each other in an axial direction of the plate-shaped electrode members, the first catalyst may only be applied to the face of an electrode member that is adjacent another electrode member within the electrode group.

Advantageously, this positioning of catalyst makes efficient use of the catalyst by positioning it close to the centre of a discharge zone in which plasma is generated in the reactor.

Where the electrode members are perforated plates, those perforate plates may be curved and/or bent. Advantageously, having curved and/or bent perforated plates can facilitate different reactor geometries.

A reactor according to the first aspect of the present disclosure may further comprise a non-conductive separator positioned between adjacent electrode members. The non-conductive separator may extend around at least a portion of the circumference of the electrode members. Advantageously, such a non-conductive separator can electrically isolate the electrode members from each other, and from the remainder of the reactor. The non-conductive separator may comprise PTFE or other electrically insulating polymer.

In a reactor according to the first aspect of the present invention in which each electrode member is a perforated plate, adjacent plate electrode members may be substantially parallel to each other and offset from each other by a distance of 1 mm or less between adjacent surfaces of the adjacent electrode members. The offset distance between adjacent electrode members may be greater than or equal to 0.01 mm. The offset distance between adjacent electrode members may be less than or equal to 1 mm. The offset distance between adjacent electrode members may be less than or equal to 0.5 mm. The offset distance between adjacent electrode members may be less than or equal to 0.25 mm. The offset distance between adjacent electrode members may be less than or equal to 0.15 mm. The offset distance between adjacent electrode members may be greater than or equal to 0.05 mm. The offset distance between adjacent electrode members may be greater than or equal to 0.09 mm. The offset distance between adjacent electrode members may be less than or equal to 0.11 mm. The offset distance between adjacent electrode members may be greater than or equal to 0.20 mm. The offset distance between adjacent electrode members may be greater than or equal to 0.4 mm. Advantageously, providing such an offset distance between adjacent plate electrode members facilitates the generation of a plasma with a voltage range of approximately 0.3 to 1.9 kV and with low electron energy, resulting in the formation of fewer plasma by-products than at higher voltages and electron energies.

In a second aspect of the present disclosure there is provided an air purification device comprising an air inlet, an air outlet and a microplasma catalytic reactor according to the first aspect of the present disclosure. In use, ambient air flows along an air pathway passing into the device via the air inlet, through the reactor and out of the device via the air outlet.

An air purification device according to the second aspect may comprise plural reactors according to the first aspect of the present disclosure positioned along the air pathway between the air inlet and the air outlet. Advantageously, including a plurality of microplasma catalytic reactors within the air purification device can allow the device to process a larger flowrate of air whilst achieving the same VOC decomposition performance.

Where there are plural microplasma catalytic reactors within the air purification device, with plural of these reactors receiving pulsed DC from a power supply, the parameters of the pulsed DC provided to a given reactor may be controllable independently of the pulsed DC provided to another such reactor. This is advantageous in allowing the different microplasma catalytic reactors with air purification device to be tailored to the decomposition of different compounds by controlling the plasma discharge within a given reactor independently of the plasma discharge within the other reactors.

Where there are plural microplasma catalytic reactors within the air purification device, those microplasma catalytic reactors may be placed in series along the air pathway between the air inlet and the air outlet. Advantageously, including a plurality of microplasma catalytic reactors in series with each other within the air purification device can increase the residence time of the ambient air in a microplasma catalytic reactor, thereby increasing the VOC conversion achieved by the air purification device.

An air purification device according to the second aspect may further comprise an adsorption unit positioned between the reactor and the air outlet along the air pathway. The adsorption unit may be configured to adsorb one or more of: the VOCs, the VOC decomposition products, and the plasma by-products, contained in an airflow out of the reactor during use of the air purification device. Advantageously, including an adsorption unit downstream can reduce the concentration of VOCs, VOC decomposition products and/or plasma by-products contained within the airflow out of the air purification device. Moreover, long-lived plasma species reaching the adsorption unit from the reactor may decompose VOCs and VOC decomposition products adsorbed onto an adsorbent within the adsorption unit, thereby increasing the lifetime of the adsorbent.

Where the air purification device comprises an adsorption unit or catalysis unit, the air purification device may be configured to be operated under a continuous storage-discharge operation in which the air flows through the reactor once. Advantageously, this can increase the flowrate of ambient air that can be introduced into the device.

Alternatively, where the air purification device comprises an adsorption unit and/or catalysis unit, the air purification device may be configured to be operated under a cyclic storage-discharge operation in which the VOCs are first stored on adsorbent/catalyst with plasma off and then oxidised by the reactive species produced with plasma on. Advantageously, this can increase the overall VOC conversion achieved and the device is more energy efficient for removal of very low concentrations of VCOs present in indoor air.

An air purification device according to the second aspect may further comprise a catalysis unit positioned between the reactor and the air outlet along the air pathway. The catalysis unit may be configured to catalyse the decomposition of one or more of: the VOCs, the VOC decomposition products, and the plasma by-products, contained in an airflow out of the reactor during use of the air purification device. Advantageously, the inclusion of a catalysis unit downstream of the reactor can allow the VOC conversion achieved by the air purification device to be further increased beyond that achieved in the reactor. Moreover, long-lived plasma species reaching the catalysis unit from the reactor may enhance decomposition of VOCs and VOC decomposition products on the surface of a catalyst in the catalysis unit, thereby further reducing the concentration of VOCs and VOC decomposition products contained within the airflow out of the air purification device. The catalysis unit may be operated at room temperature or at a higher temperature, depending on the balance of energy consumption and conversion desired.

In a third aspect of the present disclosure there is provided a method of treating ambient air to reduce a concentration of VOCs in that air using a microplasma catalytic reactor. The reactor comprises: a first electrode member comprising an electrically conducting core and a dielectric coating comprising a catalyst; and a second electrode member disposed with respect to the first electrode member. The method comprises the steps of: passing a VOC-containing ambient airflow through the reactor; applying a plasma generation voltage between the first and second electrode members to generate a plasma in the VOC-containing airflow that decomposes VOCs contained within the airflow, and generates VOC decomposition products and plasma by-products; and catalysing, using the catalyst, the decomposition of one or more of: the VOCs; the VOC decomposition products; and the plasma by-products. Advantageously, such a method of treating ambient air using a microplasma catalytic reactor is able to effectively decompose VOCs in the ambient air whilst limiting the quantity of plasma by-products and partially-oxidised VOCs released by the reactor.

The anhydrous gaseous room temperature composition of the VOC-containing ambient airflow into the reactor may be: 21% by mass O₂, and 78% by mass N₂, the balance comprising VOCs, Ar, CO2 and further comprising trace elements and molecules.

The invention includes the combination of the aspects and preferred features described except where such a combination is clearly impermissible or expressly avoided.

### SUMMARY OF THE FIGURES

Aspects, embodiments and experiments relating to the present disclosure will now be discussed with reference to the accompanying figures, in which:

FIG. 1 illustrates a microplasma catalytic reactor according to an embodiment of the first aspect of the present disclosure, having a first electrode member and a second electrode member;

FIG. 2A illustrates a microplasma catalytic reactor according to an embodiment of the first aspect of the present disclosure, having a first electrode member, a second electrode member and a third electrode member;

FIG. 2B illustrates a microplasma catalytic reactor according to an embodiment of the first aspect of the present disclosure having, plural first electrode members and plural second electrode members;

FIG. 3 illustrates the different controllable parameters of the pulsed DC provided by a power supply to an electrode member of a microplasma catalytic reactor on a voltage-time graph.

FIG. 4 illustrates an air purification device comprising plural microplasma catalytic reactors positioned in series along an air pathway through the air purification device;

FIG. 5 illustrates an air purification device comprising a HEPA filter, a microplasma catalytic reactor and a post-plasma adsorption unit;

FIG. 6A is a process flow diagram for an air purification device according to an embodiment of the second aspect of the present disclosure, the air purification device comprising a microplasma catalytic reactor and a post-plasma adsorption unit;

FIG. 6B is a process flow diagram for an air purification device according to an embodiment of the second aspect of the present disclosure, the air purification device comprising a microplasma catalytic reactor and a post-plasma catalysis unit;

FIG. 6C is a process flow diagram for an air purification device according to an embodiment of the second aspect of the present disclosure, the air purification device comprising a HEPA filter, a microplasma catalytic reactor and a post-plasma catalysis unit;

FIG. 6D is a process flow diagram for an air purification device according to an embodiment of the second aspect of the present disclosure, the air purification device comprising a microplasma catalytic reactor, a post-plasma catalysis unit and a post-plasma adsorption unit;

FIG. 7 contains chromatograms corresponding to the reactor feed and effluent streams for Comparative Example 1 in Table 3.

FIG. 8 contains chromatograms corresponding to the reactor feed and effluent streams for Example 2 in Table 3.

### DETAILED DESCRIPTION OF THE INVENTION

Aspects, embodiments and experiments relating to the present disclosure will now be discussed with reference to the accompanying figures. Further aspects and embodiments will be apparent to those skilled in the art. All documents mentioned in this text are incorporated herein by reference.

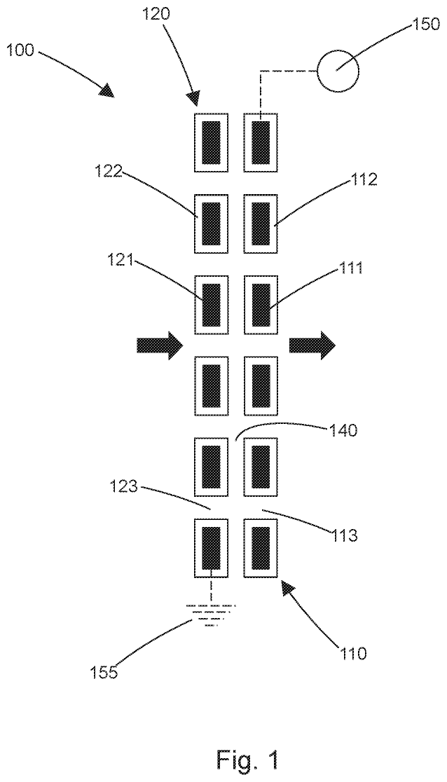

FIG. 1 illustrates a schematic of a microplasma catalytic reactor 1 according to an embodiment of the first aspect of the present disclosure. The schematic is a cross-sectional view of the catalytic reactor 100 substantially parallel to an air pathway though the reactor (illustrated by the thick arrows in FIG. 1). The reactor 1 has a first electrode member 110 and a second electrode member 120 separated by a discharge gap 140 between their adjacent surfaces. The first electrode member 110 comprises an electrically conducting core 111 and a dielectric coating 112. In the rector 100 of FIG. 1, the second electrode member 120 also comprises a respective electrically conducting core 121 and a dielectric coating 122. With both electrode members 110, 120, the dielectric coating 112, 122 of the electrode member 110, 120 fully coats the electrically conducing core 111, 112 of that electrode member 110, 120, such that no portion of the core 111, 112 is exposed on an exterior surface of that electrode member 110, 120. As is described below in greater detail, the dielectric coating of the first electrode member 110 and, optionally, the second electrode member 120 comprise one or more catalysts.

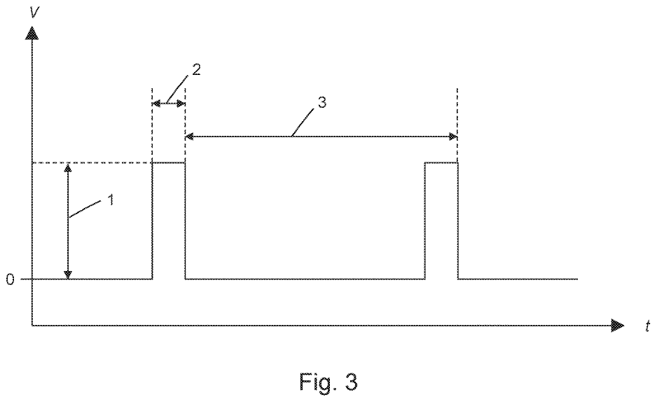

A power supply 150 is connected to the electrically conducting core 111 of the first electrode member 110, and the electrically conducting core 121 of the second electrode member 120 is connected to earth 155. Current is provided from the power supply 150 to the first electrode member such that a potential difference is provided between the electrode members 110, 120 and an electric field is generated across the discharge gap 140. The power supply 150 and the current provided to the electrode members is discussed further in relation to FIG. 3. The potential difference applied between the electrode members 110, 120 and the strength of the resulting electric field in the discharge gap 140 can be made great enough that a plasma is generated in the discharge gap 140 and in the vicinity of the electrode members 110, 120 (i.e. adjacent the surfaces of the electrode members 110, 120 not adjacent the discharge gap 140), which together form a plasma discharge zone of the reactor 100. The highly reactive species generated within the plasma are effective in decomposing VOCs contained in ambient air in the reactor. However, VOC decomposition in plasma can result in the formation of partially-oxidised VOCs (VOC decomposition products) and formation of long-lived plasma by-products such as O₃ and NOₓ. By providing a catalyst on the dielectric coating 112, 122, the decomposition of one or more of: the VOCs, the VOC decomposition products, and the plasma by-products can be catalysed in the vicinity of the plasma such as to reduce the concentration of these species in the airflow leaving the reactor 100.

Although the core 121 of the second electrode member 120 in FIG. 1 is connected to earth 155, other configurations are possible provided that a potential difference can be generated between the first electrode member 110 and second electrode member 120 in order to generate a plasma, for example, it is also possible for the cores 111, 121 of both the first electrode member 110 and the second electrode member 120 to be connected to opposite terminals of a power supply 150, or for the core 111 of the first electrode member 110 to be connected to earth 155 and the core 121 of the second electrode member 120 to be connected to a power supply 150.

In FIG. 1, both the first electrode member 110 and second electrode member 120 take the form of perforated plates, with the first electrode member 110 having a plurality of perforations 113 arranged in a regular array across its planar surface and the second electrode member 120 also having a plurality of perforations 123 arranged in a regular array across its planar surface. In the case of FIG. 1, the perforations 113 in the first electrode member 110 are aligned with the perforations 123 in the second electrode member 120 across the discharge gap 140 that separates the electrode members 110, 120. Where there is an airflow through the electrode members 110, 120 in the direction in which the perforations 113, 123 extend through the electrode members 110, 120 (as illustrated by the thick arrows in FIG. 1) the alignment of the perforations 113, 123 of adjacent electrode members 110, 120 can reduce the pressure drop across the reactor 100. However, by misaligning the perforations 113, 123, of adjacent electrode members 110, 120, the flow through the reactor 100 can be made more turbulent and the residence time and mixing of air in the discharge gap 140 can be increased. When a plasma is generated in the reactor 100 in the vicinity of the electrode members 110, 120, increasing the residence time and mixing of air in the discharge gap 140 can increase the interaction of the plasma with the catalyst disposed on the dielectric coating 112, 122, thereby increasing the VOC conversion achieved by the reactor 100. For similar reasons, it is desirable that the reactor feed airflow is controlled such that the flowrate of air through the perforations 113, 123 is uniform across the area of the electrode members 110, 120, as opposed to the flowrate through the perforations 113, 123 closer to the centre of the electrode members 110, 120 being substantially larger than the flowrate through the perforations 113, 123 closer to the edge of the electrode members 110, 120.

In a reactor such as that illustrated in FIG. 1, the cores 111, 121 of the plate electrode members 110, 120 may have a thickness of approximately 1 mm and the pitch of the apertures may be approximately 5 mm, with a 3 mm hold diameter, providing the cores 111, 112 with an open area of approximately 32.7%. The dielectric coatings 112, 122 are then uniformly applied to the cores 111, 121 and may have a thickness of approximately 200 μm, reducing the open area of the resulting electrode member 110, 120 to approximately 24.6%. The width of the discharge gap 40 (i.e. the distance between adjacent surfaces of the adjacent electrode members) is typically less than 1 mm, and preferably approximately 0.1 mm, in order to allow a plasma to be formed at a low voltage and with a low electron energy such that the formation of plasma by-products is limited (discussed below in reference to the Tests and Examples in Tables 1-3).

The cores 111, 121 of the electrode members 110, 120 can be formed from any electrically conductive material; however, it is preferable that the core comprises one or more of stainless steel, aluminium, brass, iron or copper. These metals and metal alloys have a high conductivity and are readily available. Aluminium, copper and brass are particularly advantageous in being lightweight and highly ductile in comparison to stainless steel and iron. Another factor in the selection of the material for the cores 111, 121 is the fraction of the power supplied to the electrode member that is lost as thermal energy. Of the materials listed above, stainless steel provides the highest process efficiency with respect to heat loss, with aluminium, brass, iron and copper becoming progressively less efficient. The external surfaces of the cores 111, 121 are ground prior to coating to increase surface uniformity and reduce plasma concentrated areas and/or arc formation within the reactor 100.

The dielectric coatings 112, 122 applied to the cores 111, 121 of the electrode members 110, 120 can have a substrate formed from a metal oxide such as MnO₂, Al₂O₃, CeO₂, SiO₂ and TiO₂. Alternatives to metal oxides include activated carbon and zeolites. Advantageously, these metal oxide materials and alternative materials can be provided as a substrate with a high surface area onto which to dispose the catalytic material and can also themselves act as a catalyst in the presence of a plasma. Where a dielectric coating 112, 122 is porous, the catalyst may be disposed on the substrate, at least in part, on internal surfaces of the pores of the dielectric coating. In this way, the porosity of the coating film can be controlled from a relative porous to a non-porous one by filling the pores or cracks with a catalyst. The inhomogeneity in coating film can be controlled that can alter the discharge characteristics and breakdown mechanism, and is critical to maintain the electrical, mechanical, and chemical characteristics.

In the present microplasma catalytic reactor 100, at least the first electrode member 110 has a dielectric coating 112 comprising a catalyst that catalyses the decomposition of one or more of: the VOCs, the VOC decomposition products; and plasma by-products contained within the ambient air processed by the reactor 100. If, in the reactor 100 illustrated in FIG. 1, only the first electrode member 110 comprises a catalyst, it is preferable for the first electrode member 110 to be located downstream of the second electrode member 120, as is the case in FIG. 1, because then the catalyst is located downstream of the discharge gap 140 in which plasma generated by the reactor 100 is concentrated. This then means that VOCs and plasma species are more likely to interact on a surface on which a catalyst is present, since this is the direction in which the VOCs and plasma species will travel through the reactor 100 after being generated.

The catalyst may comprise one or more of: a metal oxide, a mixed metal oxide, a noble metal, a noble metal-metal oxide composite, and a noble metal-mixed metal oxide composite. Such a catalyst may comprise one or more of Ag, Pt, Pd, Rh, Ni, Cu, Mo, Co, Mg and Ti. An incipient wetness impregnation (IWI) technique is used to dispose the selected catalyst on the surface of material forming the remainder of the dielectric coating 112, 122. The use of an IWI technique means that the macro-scale thickness and uniformity of the dielectric coating 112, 122 is not impacted by the deposition of catalyst.

Whilst the reactor 100 may only comprise a single electrode member 110 having a dielectric coating 112 comprising a catalyst, it is typical for several, or all, of the electrode members 110, 120 within the reactor 100 to have a dielectric coating 112, 122 and for each dielectric coating 112, 122 to comprise catalyst. However, the catalyst(s) that the dielectric coating 112, 122 of each electrode member 110, 120 comprises may differ between the electrode members 110, 120, such that different catalysts can be provided at different residence times within the reactor 100, i.e. the choice of catalyst for each electrode member 110, 120 can be tailored to the species present in the plasma at the position of that electrode member 110, 120 in the reactor 100.

In FIG. 1 both the first electrode member 110 and second electrode member 120 comprise a dielectric coating 112, 122 as this can help to reduce the likelihood of arcing between the electrodes and the formation of a large quantity of plasma by-products and damage to the coating caused by the arc formation.

Moreover, disposition of catalyst on the substrate of the dielectric coating 112, 122 increases the capacitance of the electrode members 110, 120, such that the reactor 100 can operate at lower current levels whilst still generating a plasma, thereby reducing the energy consumption of the reactor 100.

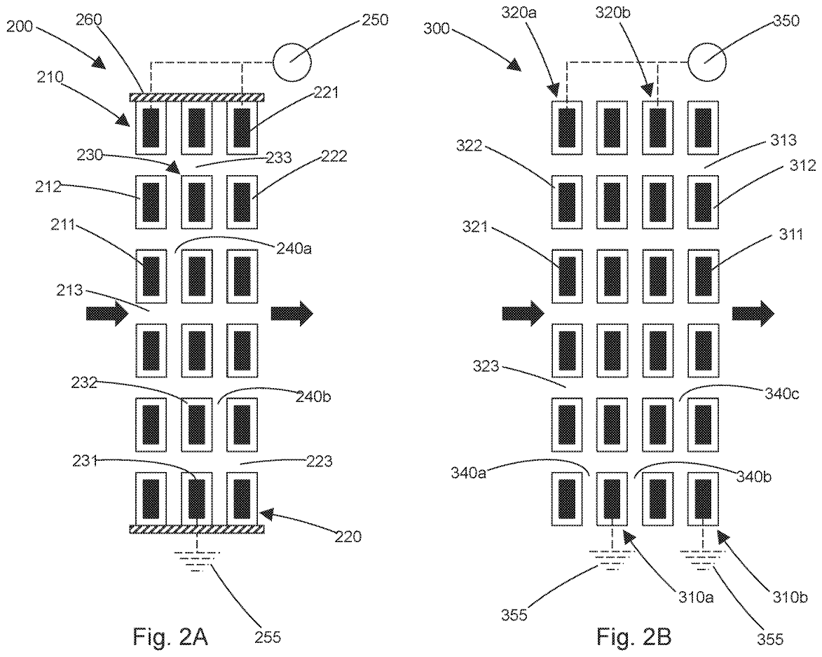

FIG. 2A illustrates a schematic of a microplasma catalytic reactor 200 according to an embodiment of the first aspect of the present disclosure. The schematic is a cross-sectional view of the catalytic reactor 200 substantially parallel to an air pathway though the reactor (illustrated by the thick arrows in FIG. 2A). The reactor 200 in FIG. 2A can be considered a modification of the reactor 100 illustrated in FIG. 1, and much of the foregoing description of FIG. 1 is applicable, mutatis mutandis, to FIG. 2A.

However, the reactor 200 in FIG. 2A differs from that illustrated in FIG. 1 in that the reactor 200 in FIG. 2A further comprises a third electrode member 230 interposed between the first electrode member 10 and the second electrode member 220. The electrode members 210, 220, 230 are spaced apart from each other such that a discharge gap 240 is provided between adjacent electrode members 210, 220, 230 as discussed in relation to FIG. 1 (i.e. the distance between the first electrode member 210 and second electrode member 220 in the reactor 200 in FIG. 2A is made greater such as to accommodate the third electrode member 230 interposed between them, with the discharge gaps 240a, 240b provided between adjacent electrode members 210, 220, 230 being within the same range of sizes as discussed in relation to the discharge gap 140 of the reactor 100 of FIG. 1). The electrode members 210, 220 230 of the reator 200 of FIG. 2A have much the same structure as set out in the forgoing description in relation to the first electrode member 110 and/or second electrode member 120 of the reactor 100 of FIG. 1. However, the catalyst(s) that the dielectric coating 212, 222, 232 of each electrode member 210, 220, 230 comprises may differ between the electrode members 210, 220, 230, such that different catalysts can be provided at different residence times within the reactor 200 such that the choice of catalyst for each electrode member 210, 220, 230 can be tailored to the species present in the plasma at the position of that electrode member 210, 220, 230 in the reactor 200.

An additional difference between the reactor 200 in FIG. 2A and the reactor 100 in FIG. 1 is the connection of the power supply 250 to the electrode members 210, 220, 230. In the reactor 100 in FIG. 1, the power supply 150 was only connected to the core 111 of the first electrode member 110, with the core 121 of the second electrode member 120 being connected to earth 155. However, with the reactor 200 of FIG. 2A, the cores 211, 221 of both the first electrode member 210 and the second electrode member 220 are connected to the power supply 250 and the core 231 of the third electrode member 230 is connected to earth 255. The connection of the power supply 150, 250 differs between FIGS. 1 and 2A because the electrode members 210, 220, 230 of the reactor 200 need to be provided with current in such a manner that there is a potential difference generated between a given electrode member and the electrode member(s) adjacent that given electrode member, such that a plasma is then generated by the resulting electric field.

It can be appreciated that by interposing the third electrode member 230 between the first and second electrode members 210, 220 as illustrated in FIG. 2A, the distance in the direction of the air pathway over which plasma is generated within the reactor 200 is increased such that, for a given flowrate of air into the reactor 200, the residence time of air within the reactor 200 is increased whilst still maintaining a discharge gap and a potential difference between adjacent electrode member 210, 220, 230 that facilitates the generation of plasma with minimal plasma by-products. Thus, the conversion of VOCs and VOC decomposition products within the reactor 200 is increased as a result of those compounds spending a greater amount of time within a plasma and proximate to a catalyst for the reactions to decompose those compounds.

An additional feature of the reactor 200 illustrated in FIG. 2A is the presence of a non-conductive separator 260 positioned between the electrode members 210, 220, 230. The non-conductive separator extends circumferentially around the electrode members 210, 220, 230 such as to isolate adjacent electrode members 210, 220, 230 from each other and from any other conductive structures within the reactor 200, for example a reactor casing. Typically, the separator 260 comprises PTFE. In order to provide adequate insulation, the separator 260 has a thickness greater than or equal to 25 μm. Generally, the separator thickness is less than or equal to 1000 μm.

FIG. 2B illustrates a schematic of a microplasma catalytic reactor 300 according to an embodiment of the first aspect of the present disclosure. The schematic is a cross-sectional view of the catalytic reactor 300 substantially parallel to an air pathway though the reactor (illustrated by the thick arrows in FIG. 2B). The reactor 300 in FIG. 2B can be considered a modification of the reactor 100 illustrated in FIG. 1, and much of the foregoing description of FIG. 1 is applicable, mutatis mutandis, to FIG. 2B.

However, the reactor 300 in FIG. 2B differs from that illustrated in FIG. 1 in that the reactor 300 in FIG. 2B comprises plural first electrode members 310 and plural second electrode members 320. Specifically, the reactor 300 in FIG. 2B comprises two first electrode members 310a and 310b and two second electrode member 320a and 320b. The pair of adjacent first electrode members 310a-b is interposed by the second electrode member 320b, and the pair of adjacent second electrode members 320a-b is interposed by the first electrode member 310a. The electrode members 310, 320 are spaced apart from each other such that a discharge gap 340 is provided between adjacent electrode members as discussed in relation to FIG. 1 (i.e. the discharge gaps 340a, 340b and 340c provided between adjacent electrode members are within the same range of sizes as discussed in relation to the discharge gap 140 of the reactor 100 of FIG. 1). The cores 321 of both the second electrode members 320a, 320b are connected to the power supply 350, whilst the cores 311 of both the first electrode members 310a, 310b are connected to earth 355. It can be appreciated that by increasing the number of first and second electrode members 310, 320 within the reactor 300 as illustrated in FIG. 2B, the distance in the direction of the air pathway over which plasma is generated within the reactor 300 in FIG. 2B is increased, such that, for a given flowrate of air, the residence time of air within the reactor 300 is increased whilst still maintaining a discharge gap and a potential difference between adjacent electrode member 310, 320 that facilitates the generation of plasma with minimal plasma by-products. Thus, the conversion of VOCs and VOC decomposition products within the reactor 300 is increased as a result of those compounds spending a greater amount of time within a plasma and proximate to a catalyst for the reactions to decompose those compounds.

In the reactor 300 in FIG. 2B, the catalyst(s) that the dielectric coating 312, 322 of each electrode member 310a-b, 320a-b comprises may differ between the electrode members, such that different catalysts can be provided at different residence times within the reactor 300, i.e. the choice of catalyst for each electrode member 310a-b, 320a-b can be tailored to the species present in the plasma at the position of that electrode member 310a-b, 320a-b in the reactor 300.

It can be appreciated that the reactor arrangements illustrated in FIGS. 2A and 2B can be expanded upon in a similar manner such as to further increase the residence time of a microplasma catalytic reactor 200, 300. For example, the reactor 300 illustrated in FIG. 2B could be scaled up such that there were three, four or five first electrode members 310 and second electrode members 320, respectively, with each pair of adjacent first electrode members 310 interposed by a second electrode member 320 and each pair of adjacent second electrode members 320 interposed by a first electrode member 310.

As discussed in relation to FIGS. 1 and 2, a power supply 150, 250, 350 is connected to one or more electrode members in the reactors 100, 200, 300 of FIGS. 1 and 2 such as to provide a current to the electrode members to which it is connected that creates a potential difference between adjacent electrode members that then generates an electric field across the discharge gap between adjacent electrode members. The potential difference applied between the electrode members and the strength of the resulting electric field in the discharge gap can be made great enough that a plasma is generated in the discharge gap and in the vicinity of the electrode members (i.e. adjacent the surfaces of the electrode members not adjacent the discharge gap), which together form a plasma discharge zone of the reactor. The energy of free electrons within the plasma generated in the reactor 100, 200, 300 influences likelihood of plasma by-product formation and also influences the conversion of VOCs contained within the plasma. Typically, the higher the free electron energy, the more plasma by-products are formed and the higher the conversion of VOCs within the reactor 100, 200, 300. Thus, a balance must be struck between plasma by-product formation and VOC conversion. Free electron energy within the plasma is strongly dependent on the electric field strength, and thus the potential difference, between adjacent electrode members. The power supply 150, 250, 350 is configured to provide pulsed DC to the electrode members to which it is connected; by providing pulsed DC to the electrode members connected to the power supply, the energy consumption of the reactor 100, 200, 300 can be reduced in comparison to using non-pulsed DC and also additional control variables for the electric field strength between adjacent electrode members, is provided.

FIG. 3 provides an illustrative voltage-time graph containing a waveform for the pulsed DC provided by the power supply to an electrode member of a microplasma catalytic reactor of the present disclosure. The pulsed DC waveform comprises pulse-ON phases, when the voltage of the power supply is non-zero, and pulse-OFF phases, when the voltage of the power supply is approximately zero. There are three parameters of the pulsed DC waveform illustrated in FIG. 3 that can be controlled in order to adjust the potential difference between adjacent electrode members in the reactor. Firstly, the voltage amplitude 1 of the pulsed DC is adjustable, the voltage amplitude 1 being the amplitude of the waveform during pulse-ON phases. Secondly, the pulse width 2 is adjustable, the pulse width 2 being the duration of a pulse-ON phase. Thirdly, the period 3 of the waveform is adjustable, the period 3 being the duration for a full pulse-ON-pulse-OFF cycle to complete. The period 3 is the reciprocal of the frequency of the pulsed DC.

The influence of these parameters of the pulsed DC on the performance of the microplasma catalytic reactor will be explained further in relation to the data in Tables 1-3 of the Examples.

In a second aspect of the present disclosure, the above-described mircoplasma catalytic reactor is incorporated into an air purification device. Such an air purification device comprises an air inlet through which VOC-containing ambient air can be provided into the reactor and an air outlet through which the air treated within the reactor can exit the air purification device. In other words, the air inlet, reactor and air outlet are all provided along an air pathway along which ambient air can flow.

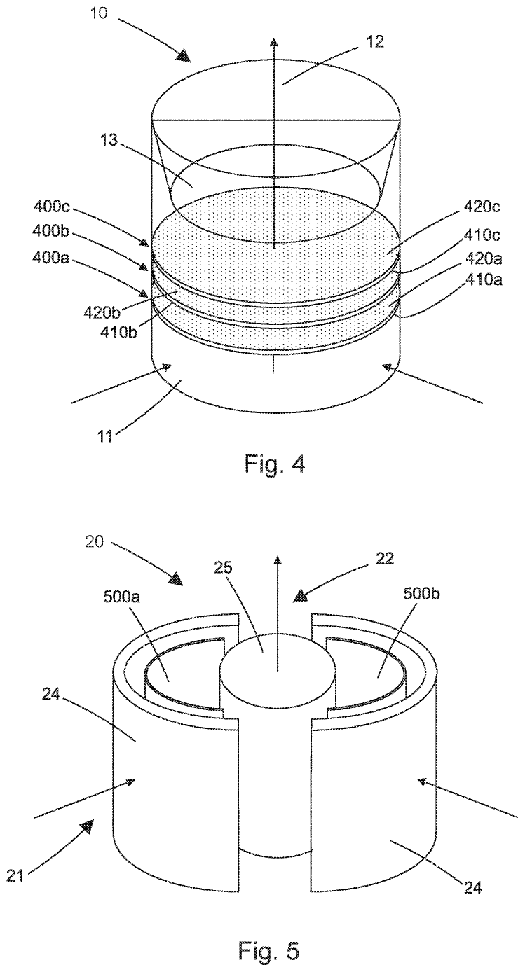

FIG. 4 illustrates an embodiment of such an air purification device 10. An air inlet 11 is provided at the base of the air purification device 10 and an air outlet 12 is provided at the top of the device 10, with three microplasma catalytic reactors 400a-c interposed between the air inlet 11 and air outlet 12 along the air pathway. The three reactors 400a-c of the device 10 are placed in series along the air pathway, such that the air exiting the first reactor 400a subsequently passes into the second reactor 400b and finally the third reactor 400c. Whilst an air purification device 10 may comprise a single reactor 400, by placing a plurality of reactors 400a-c in series with each other along the air pathway as in the device 10 of FIG. 4, the residence time of the air within the air purification device 10 for a given flowrate of air through the device 10 can be increased, in turn increasing the conversion of VOCs and VOC decomposition products that can be achieved by the device 10 as a result of those compounds spending a greater amount of time within a plasma and proximate to a catalyst for the reactions to decompose those compounds. An alternative perspective on the effect of increasing the number of reactors 400 in series along the air flow pathway is that it facilitates an increase in the flowrate of air through the device 10 without impacting the conversion of VOCs and VOC decomposition products, since the total residence time within the reactors 400a-c of the device 10 can be maintained even as the flowrate of air is increased.

Each of the reactors 400a-c in the air purification device 10 of FIG. 4 contains a respective pair of electrode members 410a-c, 420a-c that take the form of perforated plates. Each of the reactors 400a-c is provided with pulsed DC from a power supply in order to generate a plasma in the vicinity of its electrode members 410a-c, 420a-c. An advantage of having a plurality of reactors 400a-c within the air purification device 10 is that the pulsed DC provided to a given reactor 400a-c can be controlled independently of the pulsed DC provided to the other reactors 400a-c within the device 10. Accordingly, the plasma generated in a given reactor 400a-c can be controlled independently of the plasma generated in the other reactors 400a-c and can be tailored to the compounds present in the air flowing into that specific reactor 400a-c. Thus, the performance of the air purification device 10 with regards to concentration of VOCs, VOC decomposition products and plasma by-products in the air leaving the device 10 via the air outlet 12 can be improved.

The air purification device 10 in FIG. 4 further comprises an air propulsion unit 13 (e.g. a compressor, a fan, a blower) to drive an airflow along the air pathway through the device 10. In FIG. 4 the air propulsion unit 13 is located downstream of the reactors 400a-c along the air pathway such that air is sucked through the reactors 400a-c by the air propulsion device 13. However, it is also possible to provide an air propulsion unit 13 upstream of the reactors 400a-c along the air pathway. The position of the air propulsion unit 13 relative to the reactors 400a-c influences the characteristics of the airflow through the reactors 400a-c: locating the air propulsion unit 13 upstream of the reactors 400a-c results in more turbulent and higher pressure airflow through the reactors 400a-c, whilst locating the air propulsion unit 13 downstream of the reactors 400a-c provides a more laminar and lower pressure airflow through the reactors 400a-c. Although an air propulsion unit 13 is advantageous in driving an airflow through the air purification device 10, it is also possible for the device 10 to operate absent an air propulsion unit 13, for example, if the device 10 is configured and/or positioned such that an airflow is naturally convected through the device 10.

Whilst the air purification device 10 in FIG. 4 comprises three reactors 400a-c in series along the air pathway, it can be appreciated that an air purification device 10 may comprise only a single reactor 400, or may comprise plural reactors 400 that are in parallel with each other.

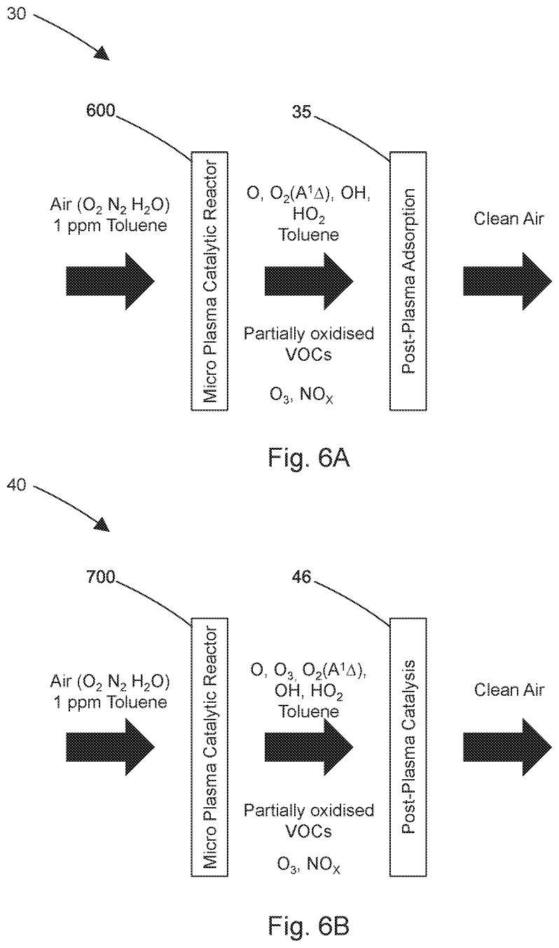

FIG. 5 illustrates an air purification device 20 with plural reactors 500a-b having an alternative arrangement to that shown in FIG. 4. In FIG. 5 each reactor 500a-b are provided as curved perforated plates that are arranged in parallel with one another, with air entering the device 20 only flowing through one of the two reactors 500a-b. Similarly to the device 10 in FIG. 4, providing a plurality of reactors 500a-b in parallel with each other allows the flowrate of air through the device 20 to be increased without impacting the conversion of VOCs and VOC decomposition products by the device 20, because the residence time within a given reactor 500a-b can be maintained even as the flowrate of air is increased because the increase volume of air can be split across the increased number of reactors 500 in parallel with each other. The air purification device 20 of FIG. 5 further comprises High-Efficiency Particulate Absorbing (HEPA) filters 24 positioned upstream of the reactors 500a-b in order to remove particulate matter such as pollen, dust, bacteria and viruses from the airflow into the device 20 prior to that air entering the reactors 500a-b. Removal of such particulate matter is desirable such that it does not interfere with the performance of the reactors 500a-b by influencing plasma generation and/or clogging the reactor. Whilst the device 20 in FIG. 5 comprises two HEPA filters 24, one corresponding to each reactor 500a-b, it is also possible to provide a device 20 where all of the airflow into the reactor(s) 500 flows through the same HEPA filter 4. Additionally, the device 20 comprises an adsorption unit 25 positioned downstream of the reactors 500a-b with respect to the air pathway through the device 20. The plasma generated within the reactors 500a-b may, depending on the operating conditions of the reactors 500a-b, lead to the formation of plasma by-products (NOₓ, O₃), particularly where the free-electron energy of the plasma is high. Additionally, VOC decomposition within the plasma generated by the reactors 500a-b can lead to the formation of harmful VOC decomposition products (partially-oxidised VOCs). Consequently, where such plasma by-products and VOC decomposition products are generated, a post-plasma adsorption unit can be used in order to adsorb these compounds from the air treated by the reactors 500a-b before it exits the air purification device 20 via the air outlet 22. Typically, adsorption units 25 within air purification devices 20 are consumable units because the adsorbent contained within them will reach its maximal loading after a certain duration of operation. However, in the present air purification device 20 the adsorbent of the adsorption unit 25 is able to be (at least partially) regenerated in-situ by long-lived plasma species (e.g., O, O₂ (A¹Δ), O₃, OH, HO₂, H₂O₂) that sustain beyond the plasma discharge zone of the reactor 500a-b. These long-lived plasma species enter the adsorption unit 25 within the airflow and decompose compounds adsorbed onto the adsorbent, with the resulting products being released from the adsorbent and exiting the adsorption unit 25 and air purification device 20 via the air outlet 22, such that the adsorbent's capacity is regenerated and the adsorption unit 25 does not require replacement as frequently as in an air purification device 20 where there is not a microplasma catalytic reactor 500 positioned upstream of the adsorption unit 25.

In FIG. 5 the adsorption unit 25 is cylindrical in form and thus the perforated plates that form the electrode members of the reactors 500a-b are curved such that they correspond to the cylindrical surface of the adsorption unit 25 that they are adjacent. In order to form electrode members having this shape, it is desirable that the electrically conducing cores of the electrode members are formed from a ductile material such as aluminium, brass or copper. The HEPA filters 24 of the device 20 are curved in a similar manner. By providing the air purification device 20 with a cylindrical form, air can be drawn into the device 20 from a wide range of directions.

Where the air purification device comprises an adsorption unit or catalysis unit, the air purification device may be configured to be operated under a continuous storage-discharge operation in which the air flows through the reactor once. Advantageously, this can increase the flowrate of ambient air that can be introduced into the device.

Alternatively, Where the air purification device comprises an adsorption unit or catalysis unit, the air purification device may be configured to be operated under a cyclic storage-discharge operation in which a proportion of the air exiting the reactor is recycled back into the reactor. Advantageously, this can increase the overall Where the air purification device comprises an adsorption unit or catalysis unit, VOC conversion achieved by the device.

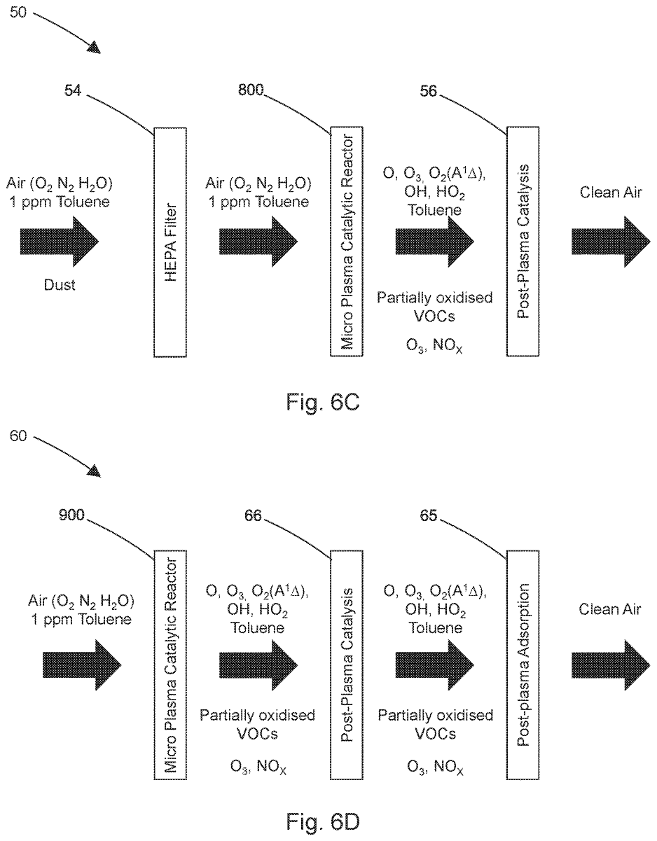

FIGS. 6A-6D provide process flow diagrams for several different configurations of air purification devices 30, 40, 50, 60 according to the present disclosure, the air purification devices 30, 40, 50, 60 comprising at least one microplasma catalytic reactor 600, 700, 800, 900 and a plurality of different additional air treatment units (adsorption units 35, 65, catalysis units 46, 56, 66, HEPA filters 54, and the like). In the flow diagrams of FIGS. 6A-6D the air purification devices 30, 40, 50, 60 are provided with a test airflow that is a humid, VOC-containing, ambient airflow containing O₂, N₂, H₂O, Toluene and other trace elements and molecules. The anhydrous gaseous room temperature composition of the VOC-containing ambient airflow into the reactors 30, 40, 50, 60 of FIGS. 6A-6D is: 21% by mass O₂, 78% by mass N₂ and 1 ppm Toluene, the balance comprising Ar, CO₂ and other trace elements and molecules. Incorporating additional air treatment units into an air purification device comprising one or more microplasma catalytic reactors 600, 700, 800, 900 is useful where the concentration of VOCs and/or the concentration of plasma by-products in the air leaving the final reactor 600, 700, 800, 900 along the air pathway through the device 30, 40, 50, 60 remains high. For example, there may be a limit on the number of reactors 600, 700, 800, 900 and/or the number of electrode members within those reactors 600, 700, 800, 900 due to factors such as the pressure drop across the reactors 600, 700, 800, 900 and the space available in the air purification device 30, 40, 50, 60 for additional reactors 600, 700, 800, 900 and/or electrode members.

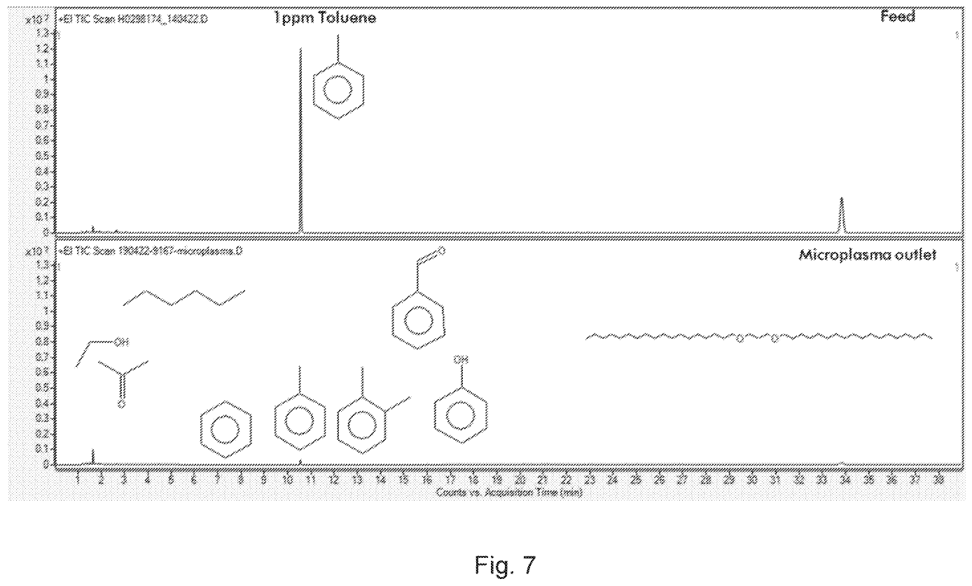

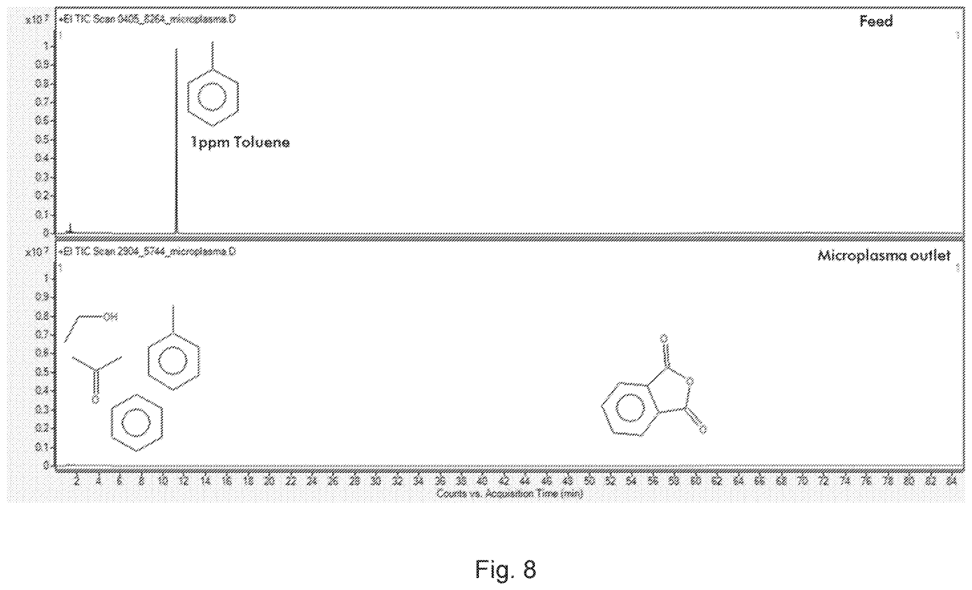

In FIG. 6A the air purification device 30 comprises a microplasma catalytic reactor 600 and a post-plasma adsorption unit 35 positioned downstream of the reactor 600 along the air pathway through the device 30 (illustrated by the thick arrows in FIG. 6A). The microplasma catalytic reactor 600 acts to decompose the toluene molecules contained in the reactor feed airflow as described above with reference to FIGS. 1-3, with the reactor effluent airflow comprising a mixture of toluene not decomposed by the reactor 30, partially oxidised VOCs produced by the decomposition of toluene, long-lived plasma species (e.g. O, O₂ (A¹Δ), OH, HO₂) and plasma by-products (e.g. O₃, NOₓ). Within the reactor effluent airflow, the concentration of toluene, the concentration and identity of the VOC decomposition products and the concentration of plasma by-products is a function of the operating conditions of the reactor 30 (as discussed above in relation to FIG. 3 and below in relation to Tables 1-3 and FIGS. 7 and 8).

The reactor effluent airflow in FIG. 6A then passes to a post-plasma adsorption unit 35. The adsorption unit 35 is provided within the device 30 in order to adsorb toluene, toluene decomposition products and plasma by-products from the air treated by the reactor 30 before the air exits the air purification device 30. As discussed in relation to FIG. 5, the adsorbent of an adsorption unit 35 positioned downstream of the microplasma catalytic reactor 600 is able to be (at least partially) regenerated in-situ by the long-lived plasma species (e.g. O, O₂ (A¹Δ), OH, HO₂) that sustain beyond the plasma discharge zone of the reactor 600 and react with the toluene, toluene decomposition products and plasma by-products that adsorb onto the adsorbent of the adsorption unit 35. The microplasma catalytic reactor 600 and post-plasma adsorption unit 35 thus act synergistically to provide a clean air stream with a reduced concentration of VOCs and that meets WHO guidelines for ozone and nitrous oxides. This clean air stream then exits the air purification device 30 shown in FIG. 6A.

In FIG. 6B the air purification device 40 comprises a microplasma catalytic reactor 700 and a post-plasma catalysis unit 46 positioned downstream of the reactor 700 along the air pathway through the device 40 (illustrated by the thick arrows in FIG. 6B). The microplasma catalytic reactor 700 acts to decompose the toluene molecules contained in the reactor feed airflow as described above with reference to FIGS. 1-3, with the reactor effluent airflow comprising a mixture of toluene not decomposed by the reactor 700, partially oxidised VOCs produced by the decomposition of toluene, long-lived plasma species (e.g. O, O₂ (A¹Δ), OH, HO₂) and plasma by-products (e.g. O₃, NOₓ). Within the reactor effluent airflow, the concentration of toluene, the concentration and identity of the toluene decomposition products and the concentration of plasma by-products is a function of the operating conditions of the reactor 700 (as discussed above in relation to FIG. 3 and below in relation to Tables 1-3 and FIGS. 7 and 8).

The reactor effluent airflow in FIG. 6B then passes to a post-plasma catalysis unit 46. The catalysis unit 46 is provided within the device 40 in order to catalyse the decomposition of one or more of the toluene, the toluene decomposition products and the plasma by-products contained within the reactor effluent airflow, such that the air leaving the catalysis unit 46 and ultimately the air purification device 40 is a clean air stream with a reduced concentration of VOCs and that meets WHO guidelines for ozone and nitrous oxides. Similarly to the device 30 in FIG. 6A, by positioning the catalysis unit 46 downstream of the microplasma catalytic reactor 700, long-lived plasma species (e.g. O, O₂ (A¹Δ), OH, HO₂) that sustain beyond the plasma discharge zone of the reactor 700 can reach the catalysis unit 46. These long-lived plasma species, in the presence of the catalyst in the catalysis unit 46 can increase the conversion of toluene and other partially oxidised VOCs on the catalyst surface and decompose plasma by-products on the catalysts surface. The catalysis unit 46 may be operated at room temperature or at a higher temperature, depending on the balance of energy consumption and conversion desired. The microplasma catalytic reactor 700 and post-plasma catalysis unit 46 thus act synergistically to provide a clean air stream with a reduced concentration of VOCs and that meets WHO guidelines for ozone and nitrous oxides. This clean air stream then exits the air purification device 40 shown in FIG. 6B.

In FIG. 6C the air purification device 50 comprises a microplasma catalytic reactor 800, a HEPA filter 54 positioned upstream of the reactor 800 along the air pathway through the device 50 (illustrated by the thick arrows in FIG. 6C) and a post-plasma catalysis unit 56 positioned downstream of the reactor 800 along the air pathway though the device 50. The HEPA filter 54 acts to remove particulate matter such as pollen, dust, bacteria and viruses from the airflow into the device 50 prior to the that air entering the reactor 800. Removal of such particulate matter is desirable such that it does not interfere with the performance of the reactor 800 by influencing plasma generation and/or clogging the reactor 800. Following this, the microplasma catalytic reactor 800 acts as described above in relation to FIGS. 6A and 6B. The reactor effluent airflow in FIG. 6C then passes to a post-plasma catalysis unit 56 that functions in the same manner as described in relation to the post-plasma catalysis unit 46 in FIG. 6B.

In FIG. 6D the air purification device 60 comprises a microplasma catalytic reactor 900, a post-plasma catalysis unit 66 positioned downstream of the reactor 900 along the air pathway through the device 60 (illustrated by the thick arrows in FIG. 6C) and a post-plasma adsorption unit 65 positioned downstream of the catalysis unit 66 along the air pathway though the device 60. This configuration can allow the air purification device 60 to continue to provide satisfactory purification of the air even when the air flowrate through the device 60 is very large, or when seeking to maximise single-pass efficiency within the device 60, as three different units are provided within the device 60 that can decompose and/or adsorb VOCs.

Although in FIG. 6D the catalysis unit 66 and the adsorption unit 65 are illustrated as distinct units, it is also possible to combine the processes of catalysis and adsorption within a single unit by doping catalyst on an adsorbent contained within the adsorption unit 65 to accelerate the oxidation of adsorbed compounds (including VOCs, VOC decomposition products and plasma by-products).

Where the air purification device comprises an adsorption unit or catalysis unit, the air purification device may be configured to be operated under a continuous storage-discharge operation in which the air flows through the reactor once. Advantageously, this can increase the flowrate of ambient air that can be introduced into the device.

Alternatively, Where the air purification device comprises an adsorption unit or catalysis unit, the air purification device may be configured to be operated under a cyclic storage-discharge operation in which a proportion of the air exiting the reactor is recycled back into the reactor. Advantageously, this can increase the overall Where the air purification device comprises an adsorption unit or catalysis unit, VOC conversion achieved by the device.

### EXAMPLES

Table 1 contains data on experiments using a microplasma reactor supplied with pulsed DC to study the formation of plasma by-products in the reactor. The reactor comprised two perforated plate electrode members having an alumina dielectric coating 200 μm thick, separated by a discharge gap of 100 μm, with no catalyst within the dielectric coating. The electrode members had a diameter of 58.4 mm and a thickness of 1.4 mm. The perforations were 2.6 mm in diameter and arranged in a regular hexagonal array with a pitch of 5 mm, providing an open area of 24.6%. Dry ambient air (relative humidity <1%) free of VOCs was supplied to the reactor at a flowrate of 5 L/min.

Tests 1-15 in Table 1 correspond to differing pulsed DC parameters, with voltage, frequency and pulse width. The concentration of ozone (O₃), nitrogen monoxide (NO) and nitrogen dioxide (NO₂) (plasma by-products) in the reactor effluent airflow was measured, alongside the current amplitude and the power consumption of the reactor. The power consumption is calculated by the time integration of the product of voltage and current pulses over one cycle by using the correlation,

$$P = \frac{1}{T} \int V ( t ) \cdot I ( t ) \cdot dt ,$$

where T is the period time, V(t) and I(t) are the applied voltage and current, respectively.

The tests in Table 1 can broadly be divided into three operational regions. There is a first region covering tests 1-9, in which the voltage of the DC power supply was within the range 0.9-1.1 kV and a plasma was generated without the formation of plasma by-products (i.e. no O₃, NO or NO₂ was formed within the reactor). The second region covers tests 10 and 12, in which pulsed DC with a voltage of 1.2 kV and frequencies of 1 kHz and 50 kHz, respectively, were supplied and plasmas were generated with the formation of O₃ but without NOₓ formation. The third region covers tests 11 and 13-15 and resulted in the formation of O₃ and NOₓ within the plasma due to the high free electron energy generated by the pulsed DC parameters of tests 11 and 13-15, which more readily caused nitrogen dissociation and lead to NOₓ formation. The presence of the second region illustrates that it is possible to selectively form O radicals within the reactor whilst suppressing the formation of N radicals.

Table 2 contains data on experiments using a microplasma reactor supplied with pulsed DC and a microplasma catalytic reactor supplied with pulsed DC to study the formation of plasma by-products in the reactors. Humid ambient air (relative humidity ~50%) free of VOCs was supplied to the reactor at a flowrate of 5 L/min. The microplasma reactor used in tests 16, 18, 20, 22, 24, 26, 28, 30, 32, 34, 36, 38, 40, 42 and 44 was the same as that used in tests 1-15 of Table 1. The microplasma catalytic reactor (used in tests 17, 19, 21, 23, 25, 27, 29, 31, 33, 35, 37, 39, 41 and 43) was the same as that used in tests 1-15 of Table 1, but with the dielectric coating further comprising MnO₂ catalyst. Manganese oxide catalyst was incorporated onto the alumina coated electrodes of the microplasma catalytic reactor by an incipient wetness impregnation technique. The catalyst precursor, manganese (II) nitrate tetrahydrate, was dissolved in water with volume equivalent to the pore volume of alumina coating. The amount of precursor in a solution was based on the mass of the metallic element (10 wt. % Mn) per mass of Al2O3 desired. Capillary action draws the precursor solution into the pores. The impregnated electrodes were dried in oven at 110° C. for 30 minutes to drive off the water, followed by calcination at 450° C. for 2 hr with heating and cooling rates of 2° C./min. This calcination process transformed the manganese nitrate precursor to manganese oxide.

Plasma discharges in humid ambient air have a strong tendency towards instabilities and thus by-product formation occurs much more readily than with dry air under the same pulsed DC parameters. First considering the microplasma reactor test results, it is evident that, under the same pulsed DC parameters, the humid air tests result in much higher concentrations of O₃, NO and NO₂ than with the dry air tests in Table 1. Moreover, for humid air, the first and second regions in Table 1 are no longer present within the range of voltages investigated in the tests of Table 2: O₃ and NOₓ formed at voltages as low as 0.7 kV. In contrast, with dry air the reactor only generated O₃ and NOₓ at voltages greater than or equal to 1.2 kV. An explanation for the behaviour seen with humid air is that the presence of water in the discharge gap alters the reaction mechanisms and rates of the reactions that form O₃ and NOₓ within the plasma. In particular, the formation of radical species O and OH within the plasma, which can act as a precursor to O₃ and NOₓ, is expected to be contributing to the greater concentration of O₃ and NOₓ in the air leaving the reactor. Many of the test in Table 2 using the microplasma catalytic reactor resulted in the O₃ concentration in the reactor effluent airflow being in excess of the WHO guidelines for O₃ exposure levels.

However, the tests in Table 2 using the microplasma catalytic reactor resulted in much lower concentrations of plasma by-products than with the microplasma reactor under the same or similar conditions. In tests 17, 19, 21, 23, 25, 27, 29, 31, 33, 35, 37, 39, 41, and 43, no O₃ was detected within the reactor effluent airflow, even up to a voltage of 1 kV. Whilst NO and NO₂ were detected in the reactor effluent airflow, the concentrations were always smaller than the concentrations seen with the microplasma reactor operating under the same or similar conditions, typically by an order of magnitude or more. A conclusion of the comparison of the tests in Table 2 using the microplasma reactor and microplasma catalytic reactor is that the MnO₂ catalyst within the dielectric layer is catalysing the decomposition of O₃, NO and NO₂ that forms within the plasma when feeding humid air into the microplasma catalytic reactor.

Table 3 contains data on examples using a microplasma catalytic reactor according to the first aspect of the present disclosure to decompose toluene contained in a reactor feed airflow and data on comparative examples using a microplasma reactor to decompose toluene contained in a reactor feed airflow. The microplasma catalytic reactor and microplasma reactor used in the examples and comparative examples, respectively, were the same as those described above in relation to Tables 1 and 2. Humid ambient air (relative humidity ~50%) containing 1 ppm of Toluene was supplied to the reactors at a flowrate of 5 L/min. The electrode members were provided with pulsed DC from a power supply, with the Examples and Comparative Examples in Table 3 covering a range of pulsed DC parameters (voltage, frequency, pulse width).

In a low-energy plasma discharge such as that generated in a microplasma reactor, toluene is oxidised to CO₂ and water by a series of reactions with electrons and active radicals (O, H, OH, N₂*). However, by supplying catalyst within the plasma discharge, an alternative reaction mechanism for toluene oxidation is provided.

The Comparative Examples of Table 3 show that a microplasma catalytic reactor can achieve toluene decomposition in excess of 70% and minimised (<1 ppb) formation of O₃, NO and NO₂ whilst operating at ambient conditions. However, FIG. 8 provides chromatograms of reactor feed and effluent airflows for comparative example 1 (0.9 kV, 25 kHz, 1 μs, 97.9% toluene conversion) and these illustrate that a range of partially-oxidised VOC compounds are present in the effluent airflow. The partially-oxidised VOC compounds present includes benzene, phenol, and ortho-xylene, which are approximately as toxic as the toluene contained in the reactor feed, and acetone and ethanol, which are less toxic. The formation of these partially-oxidised VOCs, present in concentrations up to 0.1 ppm, indicates that integration of an adsorbent bed into an air purification device downstream of the microplasma reactor would be mandatory.

Considering the Examples of Table 3, which relate to a microplasma catalytic reactor according to the present disclosure, it can be seen that improved toluene conversion with reduced plasma by-product formation is realised. Since plasma processes are non-selective, the presence of a catalyst can improve reaction selectivity by favouring certain reaction pathways. Considering O₃ formation within the plasma as an example, O₃ does not react with toluene directly, but is readily decomposed in the presence of an MnO₂ catalyst to produce oxygen radicals that have a higher oxidation activity and will react with toluene.

Moreover, the energy consumption of the microplasma catalytic reactor is approximately an order of magnitude lower than that of the microplasma reactor. This reduction in power can be attributed to both the increased capacitance of the electrode members when the alumina substrate of the dielectric coating is impregnated with catalyst (increasing the capacitance of the electrode members to 100 pF, in comparison to 20 pF with the electrode members of the microplasma reactor) and the reduced activation energy of the reaction mechanisms occurring in the presence of the catalyst. The Examples in Table 3 indicate that there is a wide operating window at 0.9 kV and 25 KHz pulsed DC, with pulse widths ranging from 0.6 to 30 μs.

Moreover, FIG. 8, which provides chromatograms of reactor feed and effluent airflows for Example 1 (0.9 kV, 25 kHz, 1 μs, 99.8% toluene conversion) illustrates the concentration (indicated by peak size) and range of partially oxidised VOCs present in the effluent airflow of the microplasma catalytic reactor are much lower. Only benzene, ethanol, acetone and phthalic anhydride are identified, and all are at very low concentrations (<0.01 ppm).

Contrasting the Examples and Comparative Examples of Table 3, it is evident that a microplasma catalytic reactor according to present disclosure can provide higher conversion, lower by-product formation (plasma by-products and partially-oxidised VOCs) and lower power consumption than a microplasma catalytic reactor operating under the same conditions.

TABLE 1

| | | Relative | Voltage/ | Frequency/ | Period/ | Pulse | Current | Power/ | O₃/ | NO/ | NO₂/ |
|---|---|---|---|---|---|---|---|---|---|---|---|
| Test | Catalyst | Humidity/% | kV | kHz | μs | Width/μs | Amplitude/A | W | ppb | ppb | ppb |
| 1 | N | <1 | 0.9 | 1 | 1000 | 4 | 7.8 | 0.2 | 0 | 0 | 0 |
| 2 | N | <1 | 0.9 | 25 | 40 | 4 | 8.1 | 4.8 | 0 | 0 | 0 |
| 3 | N | <1 | 0.9 | 50 | 20 | 4 | 7.1 | 8.6 | 0 | 0 | 0 |
| 4 | N | <1 | 1 | 1 | 1000 | 4 | 8.7 | 0.2 | 0 | 0 | 0 |
| 5 | N | <1 | 1 | 25 | 40 | 4 | 8.9 | 5.5 | 0 | 0 | 0 |
| 6 | N | <1 | 1 | 50 | 20 | 4 | 6.3 | 8.1 | 0 | 0 | 0 |
| 7 | N | <1 | 1.1 | 1 | 1000 | 4 | 9.2 | 0.2 | 0 | 0 | 0 |
| 8 | N | <1 | 1.1 | 25 | 40 | 4 | 10.0 | 5.6 | 0 | 0 | 0 |
| 9 | N | <1 | 1.1 | 50 | 20 | 4 | 7.2 | 8.6 | 0 | 0 | 0 |
| 10 | N | <1 | 1.2 | 1 | 1000 | 4 | 9.9 | 0.2 | 7.3 | 0 | 0 |
| 11 | N | <1 | 1.2 | 25 | 40 | 4 | 9.9 | 6.3 | 7.2 | 183 | 8 |
| 12 | N | <1 | 1.2 | 50 | 20 | 4 | 7.9 | 7.6 | 6.8 | 0 | 0 |
| 13 | N | <1 | 1.3 | 1 | 1000 | 4 | 9.2 | 0.1 | 7.2 | 875 | 303 |
| 14 | N | <1 | 1.3 | 25 | 40 | 4 | 7.6 | 5.4 | 7.4 | 231 | 48 |
| 15 | N | <1 | 1.3 | 50 | 20 | 4 | 6.4 | 8.1 | 7.3 | 55 | 2 |

TABLE 2

| | | Relative | Voltage/ | Frequency/ | Period/ | Pulse | Current | Power/ | O₃/ | NO/ | NO₂/ |
|---|---|---|---|---|---|---|---|---|---|---|---|
| Test | Catalyst | Humidity/% | kV | kHz | μs | Width/μs | Amplitude/A | W | ppb | ppb | ppb |
| 16 | N | 50 | 0.7 | 1 | 1000 | 4 | 0.36 | 0.013 | 2.4 | 0 | 2 |
| 17 | Y | 50 | 0.75 | 1 | 1000 | 4 | 0.33 | 0.02 | 0 | 0.2 | 0.9 |
| 18 | N | 50 | 0.7 | 25 | 40 | 4 | 0.36 | 0.332 | 15.4 | 6 | 16 |
| 19 | Y | 50 | 0.75 | 25 | 40 | 4 | 0.42 | 0.39 | 0 | 0.1 | 1.0 |
| 20 | N | 50 | 0.7 | 50 | 20 | 4 | 0.36 | 0.658 | 15.9 | 20 | 23 |
| 21 | Y | 50 | 0.75 | 50 | 20 | 4 | 0.42 | 0.78 | 0 | 0.1 | 1.1 |
| 22 | N | 50 | 0.8 | 1 | 1000 | 4 | 0.40 | 0.015 | 1.9 | 1 | 2 |
| 23 | Y | 50 | 0.8 | 1 | 1000 | 4 | 0.39 | 0.02 | 0 | 0.4 | 1.8 |
| 24 | N | 50 | 0.8 | 25 | 40 | 4 | 0.40 | 0.391 | 34.1 | 6 | 25 |
| 25 | Y | 50 | 0.8 | 25 | 40 | 4 | 0.44 | 0.44 | 0 | 0.4 | 2.1 |
| 26 | N | 50 | 0.8 | 50 | 20 | 4 | 0.40 | 0.887 | 22.8 | 32 | 73 |
| 27 | Y | 50 | 0.8 | 50 | 20 | 4 | 0.44 | 0.88 | 0 | 0.6 | 2.5 |
| 28 | N | 50 | 0.9 | 1 | 1000 | 4 | 0.44 | 0.019 | 6.4 | 2 | 3 |
| 29 | Y | 50 | 0.85 | 1 | 1000 | 4 | 0.33 | 0.02 | 0 | 0.2 | 1.1 |
| 30 | N | 50 | 0.9 | 25 | 40 | 4 | 0.45 | 0.498 | 42.8 | 13 | 55 |
| 31 | Y | 50 | 0.85 | 25 | 40 | 4 | 0.43 | 0.47 | 0 | 0.2 | 1.1 |
| 32 | N | 50 | 0.9 | 50 | 20 | 4 | 0.44 | 0.936 | 31.3 | 13 | 44 |
| 33 | Y | 50 | 0.85 | 50 | 20 | 4 | 0.46 | 0.96 | 0 | 0.1 | 1.1 |
| 34 | N | 50 | 1 | 1 | 1000 | 4 | 0.50 | 0.023 | 14.1 | 38 | 16 |
| 35 | Y | 50 | 0.9 | 1 | 1000 | 4 | 0.49 | 0.02 | 0 | 0.1 | 0.7 |
| 36 | N | 50 | 1 | 25 | 40 | 4 | 0.50 | 0.580 | 50.1 | 32 | 121 |
| 37 | Y | 50 | 0.9 | 25 | 40 | 4 | 0.48 | 0.51 | 0 | 0.5 | 0.7 |
| 38 | N | 50 | 1 | 50 | 20 | 4 | 0.42 | 0.838 | 24.8 | 29 | 33 |
| 39 | Y | 50 | 0.9 | 50 | 20 | 4 | 0.44 | 0.88 | 0 | 0.2 | 0.7 |
| 40 | N | 50 | 1.1 | 1 | 1000 | 4 | 0.82 | 0.020 | 0 | 942 | 162 |
| 41 | Y | 50 | 1 | 1 | 1000 | 4 | 0.39 | 0.02 | 0 | 4.6 | 2.5 |
| 42 | N | 50 | 1.1 | 25 | 40 | 4 | 0.38 | 0.445 | 0 | 4233 | 340 |
| 43 | Y | 50 | 1 | 25 | 20 | 4 | 0.45 | 0.90 | 0 | 2.6 | 9.6 |
| 44 | N | 50 | 1.1 | 50 | 20 | 4 | 0.43 | 0.941 | 0 | 2237 | 38 |

TABLE 3

| | Voltage/ | Frequency/ | Period/ | Pulse | Current | Power/ | O₃/ | NO/ | NO₂/ | Toluene |
|---|---|---|---|---|---|---|---|---|---|---|
| | kV | kHz | μs | Width/μs | Amplitude/A | W | ppb | ppb | ppb | Conversion/% |
| Example 1 | 0.9 | 25 | 40 | 0.6 | 0.439 | 0.55 | 0 | 0 | 0.1 | 99.8 |
| Example 2 | 0.9 | 25 | 40 | 1 | 0.461 | 0.75 | 0 | 0.1 | 0.3 | 99.8 |
| Example 3 | 0.9 | 25 | 40 | 4 | 0.485 | 0.46 | 0 | 0.1 | 0.6 | 99.8 |
| Example 4 | 0.9 | 25 | 40 | 10 | 0.49 | 0.26 | 0 | 0.1 | 0.9 | 100.0 |
| Example 5 | 0.9 | 25 | 40 | 30 | 0.394 | 0.10 | 0 | 0 | 0.4 | 99.3 |
| Comparative | 0.9 | 25 | 40 | 1 | 8.5 | 9.5 | 0 | 0 | 0.5 | 97.9 |
| Example 1 | | | | | | | | | | |
| Comparative | 0.9 | 25 | 40 | 4 | 8.0 | 7.6 | 0 | 0.8 | 0.5 | 73.0 |
| Example 2 | | | | | | | | | | |
| Comparative | 0.9 | 25 | 40 | 30 | 6.9 | 4.0 | 0 | 0 | 0.9 | 98.5 |
| Example 3 | | | | | | | | | | |
| Comparative | 0.9 | 1 | 1000 | 1 | 8.6 | 0.37 | 0 | 0 | 0.6 | 99.3 |
| Example 4 | | | | | | | | | | |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A microplasma catalytic reactor for treatment of ambient air by decomposition of VOCs in the ambient air, the reactor comprising:
a first electrode member comprising an electrically conducting core and a dielectric coating; and
a second electrode member disposed with respect to the first electrode member so as to generate a plasma between the first and second electrode members on the application of a plasma generation voltage between the first and second electrode members such that, in use, VOCs in the ambient air in the reactor are decomposed to form VOC decomposition products, and plasma by-products are formed;
wherein the dielectric coating further comprises a catalyst that catalyses the decomposition of one or more of:
the VOCs;
the VOC decomposition products; and
the plasma by-products.

2. The reactor according to claim 1, wherein the second electrode member comprises an electrically conducting core and a dielectric coating.

3. The reactor according to claim 1, wherein:
the dielectric coating is porous; and
the catalyst is disposed, at least in part, on internal surfaces of the pores of the dielectric coating.

4. The reactor according to claim 1, wherein the dielectric coating comprises metal oxide, optionally one or more of MnO₂, Al₂O₃, CeO₂, SiO₂ and TiO₂.

5. The reactor according to claim 1, wherein:
the reactor comprises a plurality of first electrode members and a plurality of second electrode members;
each pair of adjacent first electrode members is interposed by a second electrode member; and
each pair of adjacent second electrode members is interposed by a first electrode member.

6. The reactor according to claim 1, wherein:
the reactor further comprises a power supply connected to each first electrode member or each second electrode member; and
the power supply is configured to deliver pulsed DC to each electrode member to which it is connected such as to generate a plasma between a first electrode member and an adjacent second electrode member.

7. The reactor according to claim 6, wherein the power supply is configured to deliver pulsed DC to each electrode member to which it is connected, the pulsed DC received by each electrode member:
generating an electric field strength with applied voltage greater than or equal to 0.3 kV and less than or equal to 1.9 kV between that electrode member and an adjacent electrode member;
having a pulse frequency greater than or equal to 0.5 kHz and less than or equal to 100 kHz; and
having a pulse width greater than or equal to 0.05 μs and less than or equal to 50 μs.

8. The reactor according to claim 1, wherein each electrode member is a perforated plate.

9. The reactor according to claim 8, wherein adjacent plate electrode members are substantially parallel to each other and offset from each other by a distance of 1 mm or less between adjacent surfaces of the adjacent plate electrode members.

10. An air purification device comprising:
an air inlet;
an air outlet; and
a reactor according to claim 1;
wherein, in use, ambient air flows along an air pathway passing into the device via the air inlet, through the reactor, and out of the device via the air outlet.

11. The air purification device according to claim 10, positioned along the air pathway between the air inlet and the air outlet.

12. The air purification device according to claim 11, wherein the plural reactors are positioned in series along the air pathway between the air inlet and the air outlet.

13. The air purification device according to claim 12, wherein:
the air purification device further comprises an adsorption unit positioned between the reactor and the air outlet along the air pathway; and
the adsorption unit is configured to adsorb one or more of:
the VOCs;
the VOC decomposition products; and
the plasma by-products;
contained in an airflow out of the reactor during use of the air purification device.

14. The air purification device according to claim 12, wherein:
the air purification device further comprising a catalysis unit positioned between the reactor and the air outlet along the air pathway; and
the catalysis unit is configured to catalyse the decomposition of one or more of:
the VOCs;
the VOC decomposition products; and
the plasma by-products;
contained in an airflow out of the reactor during use of the air purification device.

15. A method of treating ambient air to reduce a concentration of VOCs in that air using a microplasma catalytic reactor, the reactor comprising:
a first electrode member comprising an electrically conducting core and a dielectric coating comprising a catalyst; and
a second electrode member disposed with respect to the first electrode member;
wherein the method comprises the steps of:
passing a VOC-containing ambient airflow through the reactor;
applying a plasma generation voltage between the first and second electrode members to generate a plasma in the VOC-containing airflow that decomposes VOCs contained within the airflow, and generates VOC decomposition products and plasma by-products; and
catalysing, using the catalyst, the decomposition of one or more of:
the VOCs;
the VOC decomposition products; and
the plasma by-products.
